(19) United States
(12) Patent Application Publication
(10) Pub. No.: US 2023/0287514 A1
(43) Pub. Date: Sep. 14, 2023

(54) METHOD FOR DETECTING LUNG CANCER USING MICRORNA EXPRESSION AND METABOLOMICS

(71) Applicants: THE GOVERNORS OF THE UNIVERSITY OF ALBERTA, Edmonton (CA); ALBERTA HEALTH SERVICES, Edmonton (CA)

(72) Inventors: Eric L.R. BÉDARD, Edmonton (CA); Wilson ROA, Edmonton (CA)

(73) Assignees: THE GOVERNORS OF THE UNIVERSITY OF ALBERTA, Edmonton (CA); ALBERTA HEALTH SERVICES, Edmonton (CA)

(21) Appl. No.: 18/180,715

(22) Filed: Mar. 8, 2023

Related U.S. Application Data
(60) Provisional application No. 63/319,425, filed on Mar. 14, 2022.

(51) Int. Cl. C12Q 1/6886

(52) U.S. Cl. CPC C12Q 1/6886; C12Q 2600/158; C12Q 2600/178

(57) ABSTRACT

A method of detecting lung cancer in a subject involves: a) determining in a serum sample, the expression level of one or more miRNAs; b) determining in a urine sample, the concentration of one or more metabolites; c) comparing the expression level of the one or more miRNAs with the expression level of the one or more miRNAs in a normal control; d) comparing the concentration of the one or more metabolites with the concentration of the one or more metabolites in a normal control; e) determining whether the subject has lung cancer in accordance with the result of steps (c) and (d); wherein a difference in the expression level of the one or more miRNAs relative to the expression level of the normal control, and a difference in the concentration of the one or more metabolites relative to the concentration of the normal control, are indicative of lung cancer; and f) treating the subject with a cancer management program based on the determination in step (e).

### CROSS REFERENCE TO RELATED APPLICATION

This application claims the benefit of priority to U.S. Provisional Pat. Application Serial No. 63/319,425, filed Mar. 14, 2022.

### FIELD OF THE INVENTION

The present invention relates to microRNAs and metabolites associated with lung cancer and methods of using the microRNAs and metabolites as biomarkers for detection of lung cancer and for its treatment.

### BACKGROUND OF THE INVENTION

Lung cancer, which is characterized by uncontrolled cell growth in tissues of the lung, is the leading cause of cancer-related death in men and the second most common in women after breast cancer. Cancer originating from lung cells is regarded as a primary lung cancer and can start in the bronchi or in the alveoli. Cancer may also metastasize to the lung from other parts of the body. The two main types of lung cancer are non-small cell lung carcinoma (NSCLC) and small cell lung carcinoma (SCLC). SCLC is aggressive and refers to a form of bronchogenic carcinoma seen in the wall of a major bronchus, usually in a middle-aged person with a history of tobacco smoking. By the time most patients are diagnosed with either type, the cancer has metastasized to other parts of the body. NSCLC grows slower than SCLC and comprises all the lung carcinomas except small cell carcinoma, and includes adenocarcinoma of the lung, large cell carcinoma, and squamous cell carcinoma. NSCLC is the leading cause of cancer-related mortality worldwide. Overall survival remains poor at approximately 19%, with most patients presenting with advanced or metastatic disease. Outcomes in stage I disease however, may be as high as 88% at 10 years.

Early detection of lung cancer through screening programs may improve survival. A 20% reduction in lung cancer mortality was observed with low-dose computed tomography screening versus chest x-ray. Previous studies have shown excellent clinical outcomes in patients treated for screening-detected lung cancers. However, low-dose computed tomography screening has a high false-positive rate, with over 96% of screen-detected lesions representing false positive results. Low-dose computed tomography screening can thus lead to invasive interventions (needle biopsy and/or surgery) for many patients with benign lesions, leading to patient morbidity and significant health care costs. Additionally, cumulative radiation exposure from repeated scans may increase the risk of developing cancers. Therefore, additional complimentary screening tools to improve the specificity and cost-efficiency of low-dose computed tomography screening would be highly valuable.

Advances in molecular genetics have enabled the identification of genetic markers which are associated with cancer and may serve as useful tools for diagnostic or prognostic methods. MicroRNAs (miRNAs) are a class of single-stranded non-coding ribonucleic acid molecules of about 19-25 nucleotides in length which are involved in cellular proliferation, differentiation, apoptosis and oncogenesis (Calin et al., 2006). Their role in lung cancer is an area of active research and they rank among the top biomarker candidates (Eder et al., 2005; Weiss et al., 2008; Berindan-Neagoe etal., 2014). Previous studies have investigated the utility of sputum miRNAs for lung cancer detection (Roa et al., 2012; Shen et al., 2014; Kim et al., 2015; Xing et al., 2015; Razzak et al., 2016). Serum and plasma samples are alternative bio-specimens for miRNA profiling that avoid the difficulty of poor sample quality which can commonly occur with sputum sampling (Bianchi et al., 2011; Wozniak et al., 2015; Boeri et al., 2011; Sozzi et al., 2014; Wang et al., 2015; Nadal et al., 2015). Studies have investigated large miRNA panels for lung cancer diagnosis, including 34 serum miRNAs (area under curve [AUC] =0.89) (Bianchi et al., 2011), 24 plasma miRNAs (AUC=0.94) (Wozniak et al., 2015), 13 serum miRNAs (AUC=0.97) (Boeri et al., 2011), and 24 plasma miRNAs for identifying risk (AUC=0.85) and diagnosis (AUC=0.88) (Sozzi et al., 2014). An ongoing challenge has been to reduce the size of the miRNA panel without compromising sensitivity or specificity. Wang et al. (2015) used a panel of five serum miRNAs (miR-483-5p, miR-193a-3p, miR-25, miR-214 and miR-7) to differentiate NSCLC and healthy controls with AUC=0.823, 89% sensitivity and 68% specificity. Nadal et al. (2015) used a panel of four serum miRNAs (miR-193b, miR-301, miR-141 and miR-200b) to discriminate lung cancer cases and healthy controls with AUC=0.993, 97% sensitivity and 96% specificity. The commonality of these studies is the relatively large number of serum miRNAs used for profiling, resulting in high costs and practical challenges for their application to clinical diagnosis and screening programs.

Metabolomics has attracted attention owing to its closest relevance to phenotypes as compared with genome and proteome, and recent technological advances in nuclear magnetic resonance and mass spectrometry in obtaining more data from each biological sample (Emwas et al., 2013). The challenge is immense for bio-specimens. For example, urine contains about 2500 metabolites (Moldovan et al., 2014). However, only about 220 metabolites have been identified and quantitated by NMR or liquid chromatography-mass spectrometry (Bouatra et al., 2013). In cancer diagnosis by urine NMR metabolomics, only tens of metabolites (<100) can be quantified by untargeted metabolomics (Carrola et al., 2011). In significantly overlapped NMR spectra, these concentrations of tens of metabolites from untargeted metabolomics are complex challenges. Metabolomics may thus provide objective comprehensive analysis of low-molecular weight metabolites in biological samples, and an understanding of the biological pathways involved in the onset and progression of diseases, providing valuable insights into the molecular mechanisms of pathological processes.

Altered metabolism is currently considered as an emerging cancer hallmark, with cancer cells exhibiting a distinct metabolic behavior and impacting on systemic metabolism. Therefore, assessing metabolic changes in bio-fluids such as plasma/serum and urine can relate to tumor onset and progression and has been embraced as a promising tool in cancer detection and screening (Klupczynska et al., 2017; Mazzone et al., 2016; Carrola et al., 2011; Rocha et al., 2011). NMR exhibits high reproducibility of metabolite measurement, and requires minimum sample preparation as compared with other techniques such as mass spectrometry (Beckonert et al., 2007). Metabolomics by ¹H-NMR spectroscopy has been applied for the identification of different biomarkers in lung, breast, gastric, prostate, and breast cancers (Klupczynska et al., 2017; Mazzone et al., 2016; Carrola et al., 2011; Rocha et al., 2011). NMR may thus provide a faster, cheaper diagnosis platform of lung cancer than miRNA profiling by RT-qPCR (Beckonert et al., 2007). Most NMR metabolomics investigate metabolite profiling in plasma/serum. Carrola et al. (2011) indicates that the NMR metabolomics profiles of urine consistently differ between lung cancer patients and healthy subjects. Rocha et al. (2011) demonstrates the potential of NMR metabolomics to detect metabolic signatures in plasma at initial lung cancer stages and assesses the influence of possible confounders.

While each of the foregoing techniques may be adequate for their intended purposes, some require surgical techniques or radiation which can be traumatic to the patient, require complex equipment, special medical expertise, or time-consuming analysis of volumes of data to conduct the procedures and to make the diagnoses. Waiting for test results can also be challenging and stressful for patients.

Accordingly, there is a need for improved methods of detecting lung cancer since current techniques have not been shown to improve lung cancer survival, and usually are not definitive, costly, rapid, and have psychological or physical repercussions in the event that false-positive or false-negative results are obtained.

### SUMMARY OF THE INVENTION

The present invention relates to microRNAs and metabolites associated with lung cancer and methods of using the microRNAs and metabolites as biomarkers for detection of lung cancer and for its treatment.

In one aspect, the invention comprises a method of detecting lung cancer in a subject comprising the steps of:
a) determining in a serum sample, the expression level of one or more miRNAs;
b) determining in a urine sample, the concentration of one or more metabolites;
c) comparing the expression level of the one or more miRNAs with the expression level of the one or more miRNAs in a normal control;
d) comparing the concentration of the one or more metabolites with the concentration of the one or more metabolites in a normal control;
e) determining whether the subject has lung cancer in accordance with the result of steps (c) and (d); wherein a difference in the expression level of the one or more miRNAs relative to the expression level of the normal control, and a difference in the concentration of the one or more metabolites relative to the concentration of the normal control, are indicative of lung cancer; and
f) treating the subject with a cancer management program based on the determination in step (e).

In one embodiment, only steps (a), (c), (e), and (f) are conducted. In one embodiment, only steps (b), (d), (e), and (f) are conducted.

In one embodiment, the lung cancer comprises non-small cell lung carcinoma. In one embodiment, the one or more miRNAs comprise miR-21 and miR-223. In one embodiment, the step of determining the expression level of the one or more miRNAs comprises a real-time reverse transcription-quantitative polymerase chain reaction (RT-qPCR) assay. In one embodiment, the method further comprises using Caenorhabditis elegans miR-39-5p as a control.

In one embodiment, the one or more metabolites comprise 4-methoxyphenylacetic acid. In one embodiment, the step of determining the concentration of the one or more metabolites comprises ¹H-nuclear magnetic resonance spectroscopy. In one embodiment, the method further comprises using one or more of 4-methoxyphenylacetic acid, citrate, creatine ribosome, creatinine, choline, and n-acetyl-neuraminic acid as quantified in normal urine as a control.

In one embodiment, the steps (c) and (d) comprise statistical analysis selected from binary logistic regression, receiver operating characteristic curve, or both.

In one embodiment, the steps (c) and (d) comprise a mathematical algorithm to express oncogenic and cancer-suppressive characteristics of the miRNAs, metabolites, or both based on their biological functional pathways.

In one embodiment, the method further comprises assessing one or more parameters selected from demographic data, clinical characteristics, functional status, social/occupational history, diagnostic imaging scans, pathology reports, pulmonary function test results, previous medical and surgical history, age, gender, history of smoking, and presence of chronic obstructive pulmonary disease.

In one embodiment, the method further comprises assessing the subject’s response to lung cancer treatment comprising determining the expression levels of the miRNAs and the concentrations of the metabolites prior to treatment and after treatment, comparing the expression levels of the miRNAs and the concentrations of the metabolites in a normal control, and predicting a response if there is a difference in the levels.

In one aspect, the invention comprises a method of analyzing for a marker indicative of lung cancer comprising:
a) obtaining a sample of serum, urine, or both from a subject suspected of having lung cancer;
b) determining in the serum sample, the expression level of one or more miRNAs;
c) determining in a urine sample, the concentration of one or more metabolites;
d) comparing the expression level of the one or more miRNAs with the expression level of the one or more miRNAs in a normal control;
e) comparing the concentration of the one or more metabolites with the concentration of the one or more metabolites in a normal control; and
f) determining whether the subject has lung cancer in accordance with the result of steps (d) and (e); wherein a difference in the expression level of the one or more miRNAs relative to the expression level of the normal control, and a difference in the concentration of the one or more metabolites relative to the concentration of the normal control, are indicative of lung cancer.

In one aspect, the invention comprises a method for developing a tool for detecting lung cancer in a subject comprising the steps of:
training a neural network with a first data set comprising known data having known values for expression levels of miRNAs and concentrations of metabolites in normal controls and lung cancer subjects;
validating the neural network by providing a second data set comprising known data having known values for expression levels of miRNAs and concentrations of metabolites in normal controls and lung cancer subjects to the neural network; and
testing the neural network by providing a third data set comprising known data having known values for expression levels of miRNAs and concentrations of metabolites in normal controls and lung cancer subjects to the neural network for analysis and determination of a score ranging between 0 and 1;
wherein the determined score near 0 indicates a low likelihood of lung cancer, or the determined score near 1 indicates a high likelihood of lung cancer.

In one aspect, the invention comprises a neural network tool developed by the above method.

In one aspect, the invention comprises a system for detecting lung cancer in a subject comprising:
a computer device for inputting a data set comprising one or more values for expression levels of miRNAs and one or more concentrations of metabolites in a subject;
a neural network trained to detect lung cancer for determining a score ranging between 0 and 1 and indicative of the likelihood of lung cancer on the basis of the data set;
a display device for displaying the determined score in a human-readable form, wherein the determined score near 0 indicates a low likelihood of lung cancer or the determined score near 1 indicates a high likelihood of lung cancer.

In one aspect, the invention comprises a method for detecting lung cancer in a subject comprising:
obtaining a sample of serum, urine, or both from the subject;
determining in the serum sample, the expression level of one or more miRNAs;
determining in a urine sample, the concentration of one or more metabolites;
inputting, using a computer device, a data set comprising one or more values representing the expression level of the one or more miRNAs and the concentration of the one or more metabolites;
applying a neural network to the one or more values, wherein the neural network was trained to detect lung cancer for determining a score ranging between 0 and 1 and indicative of the likelihood of lung cancer on the basis of the data set;
displaying, using a display device, the determined score in a human-readable form, wherein the determined score near 0 indicates a low likelihood of lung cancer or the determined score near 1 indicates a high likelihood of lung cancer; and
treating the subject with a cancer management program based on the determined score.

Additional aspects and advantages of the present invention will be apparent in view of the description, which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings. In the drawings:

FIG. 1 is a flow diagram showing one embodiment of a method for detecting non-small cell lung cancer in a subject using serum miRNAs.

FIGS. 2A-B are box-plots of miR-21 (FIG. 2A) and miR-223 (FIG. 2B) for lung cancer (LC) and healthy control (HV) subjects, showing median expression of miR-21 and miR-223 with the respective 95% Cl.

FIGS. 3A-B are receiver operating characteristic curves for genomRAT (FIG. 3A) and genomVAR (FIG. 3B) for miRNA profiling of 45 subjects (28 non-small cell lung cancer and 17 healthy control subjects), showing an AUC of 0.876 and 0.912, respectively.

FIGS. 4A-B are box-plots of 4-methoxyphenylacetic acid (FIG. 4A) and citrate (FIG. 4B) for lung cancer (LC) and healthy control (HV) subjects, showing median expression of 4-MPLA and citrate with the respective 95% Cl.

FIGS. 5A-B show receiver operating characteristic curves for 4-methoxyphenylacetic acid (FIG. 5A) and citrate (FIG. 5B) for 56 subjects (32 non-small cell lung cancer and 24 healthy control subjects).

FIGS. 6A-B show receiver operating characteristic curves for comboVAR (FIG. 6A) and comboRAT (FIG. 6B) for 35 subjects (21 non-small cell lung cancer and 14 healthy control subjects).

FIGS. 7A-B show receiver operating characteristic curves of urine metabolite profiling of metabVAR and metabRAT for 56 subjects (32 non-small cell lung cancer and 24 healthy control subjects), showing an AUC of 0.827 and 0.854, respectively.

FIG. 8 shows a receiver operating characteristic curve for combination models of comboVAR for 35 subjects indicating an AUC of 1.00 (21 non-small cell lung cancer and 14 healthy control subjects).

### DETAILED DESCRIPTION OF PREFERRED EMBODIMENTS

Before the present invention is described in further detail, it is to be understood that the invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention, subject to any specifically excluded limit in the stated range. Where the stated range includes one or both limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

It must be noted that as used herein and in the appended claims, the singular forms “a,” “an,” and “the” include plural referents unless the context clearly dictates otherwise.

The present invention relates to microRNAs and metabolites associated with lung cancer and methods of using the microRNAs and metabolites as biomarkers for detection of lung cancer and for its treatment.

In one embodiment, the invention comprises a method of detecting lung cancer in a subject comprising the steps of:
a) determining in a serum sample, the expression level of one or more miRNAs;
b) determining in a urine sample, the concentration of one or more metabolites;
c) comparing the expression level of the one or more miRNAs with the expression level of the one or more miRNAs in a normal control;
d) comparing the concentration of the one or more metabolites with the concentration of the one or more metabolites in a normal control;
e) determining whether the subject has lung cancer in accordance with the result of steps (c) and (d); wherein a difference in the expression level of the one or more miRNAs relative to the expression level of the normal control, and a difference in the concentration of the one or more metabolites relative to the concentration of the normal control, are indicative of lung cancer; and
f) treating the subject with a cancer management program based on the determination in step (e).

In one embodiment, the steps (c) and (d) comprise a mathematical algorithm to express oncogenic and cancer-suppressive characteristics of the miRNAs, metabolites, or both based on their biological functional pathways.

As used herein, the term “lung cancer” means non-small cell lung carcinoma (abbreviated as “NSCLC”) referring to a group of lung cancers comprising all the carcinomas except small cell carcinoma, and including adenocarcinoma of the lung, large cell carcinoma, and squamous cell carcinoma. As used herein, the terms “cancer” and “carcinoma” are synonymous and may be used interchangeably.

As used herein, the term “subject” refers to any member of the animal kingdom. In one embodiment, a subject is a human patient. In one embodiment, a subject is an adult patient 18 years of age or older.

As used herein, the term “biological sample” means a sample of blood, plasma, serum, or urine. As used herein, the term “plasma” means the liquid component of blood that holds the blood cells of whole blood in suspension. Plasma makes up about 55% of total blood volume, and is composed mostly of water (95% by volume), dissolved proteins, glucose, clotting factors, mineral ions, hormones, carbon dioxide (plasma being the main medium for excretory product transportation), and oxygen. As used herein, the term “serum” means the clear liquid which is separated from clotted blood (i.e., plasma without clotting factors). Centrifugation and filtration are commonly used to separate red blood cells, white blood cells, and platelets from plasma or serum. As used herein, the term “urine” means the waste fluid produced by the kidneys. Any one or more of blood, plasma, serum, and urine may be suitable for testing in the present invention.

As used herein, the term “microRNA” (abbreviated as “miRNA”) means a class of non-coding RNA molecules of about 19-25 nucleotides derived from endogenous genes which act as post-transcriptional regulators of gene expression. They are processed from longer (ca 70-80 nt) hairpin-like precursors termed pre-miRNAs by the RNAse III enzyme Dicer. miRNAs assemble in ribonucleoprotein complexes termed “miRNPs” and recognize their target sites by antisense complementarity, thereby mediating down-regulation of their target genes. Near-perfect or perfect complementarity between the miRNA and its target site results in target mRNA cleavage, whereas limited complementarity between the miRNA and the target site results in translational inhibition of the target gene.

As used herein, the term “metabolite” means a substance essential to the metabolism of a subject or a particular metabolic process.

The microRNAs and metabolites disclosed herein are predictive, diagnostic, and prognostic biomarkers of lung cancer and may be of therapeutic value since certain miRNAs and metabolites may be correlated with certain cancers. As used herein, the term “biomarker” or “marker” refers to a biological molecule, such as, for example, a miRNA, metabolite, and the like, whose presence or concentration can be detected and correlated with a known condition, such as lung cancer. The biomarker or marker can be utilized as part of a predictive, prognostic or diagnostic process in healthy conditions or disease states, or which, alternatively, can be used in methods for identifying a useful treatment or prevention therapy.

In one embodiment, a biological sample is acquired from a subject. The sample can be of any biological tissue or fluid, but preferably a fluid to be non-invasive in contrast to conventional clinical practice which requires invasive sampling. In one embodiment, the sample is selected from one or more of blood, plasma, serum, and urine. In one embodiment, the sample is serum, urine, or both. Additional subject information can be incorporated into the methods described herein to assess the subject for lung cancer. Additional subject information includes, but is not limited to, demographic data, clinical characteristics, functional status, social/occupational history, diagnostic imaging scans, pathology reports, pulmonary function test results, previous medical and surgical history, age, gender, history of smoking, presence of chronic obstructive pulmonary disease, and the like. As used herein, the term “assess” or “assessing” includes any form of measurement, and includes determining if an element is present or not. The terms “determining,” “measuring,” “evaluating,” “assessing” and “assaying” can be used interchangeably and can include quantitative and/or qualitative determinations.

The miRNAs and metabolites are detected in the biological sample obtained from the subject. In one embodiment, one or more miRNAs are detected in serum. MicroRNAs are relatively stable and well preserved in serum. In one embodiment, the miRNAs comprise miR-21 and miR-223. In one embodiment, the expression level of the one or more miRNAs is determined. As used herein, the term “level” refers to a determined expression level of the miRNA. The term includes a determined level of the miRNA as compared to a reference (for example, a control, a reference biomarker, a baseline, or the like). In one embodiment, determining the expression level of the miRNA comprises the use of a real-time reverse transcription-quantitative polymerase chain reaction (RT-qPCR) assay, or other suitable technique. In one embodiment, the method further comprises using synthetic Caenorhabditis elegans miR-39-5p (abbreviated as “cel-miR-39”) as an exogenous control for spike-in during RNA extraction procedures and subsequent normalization in RT-qPCR assays. Use of cel-miR-39 ensures effective RNA extraction, cDNA synthesis, and PCR amplification, and avoids the issue of frequent hemolysis that can occur with miR-16 as an endogenous control, and the problem of extensive RNAse-mediated degradation with using U6 snRNA (Navarro et al., 2011; Kirschner et al., 2011; Xiang et al., 2014).

In one embodiment, one or more metabolites are detected in urine. In one embodiment, the metabolite comprises 4-methoxyphenylacetic acid. As used herein, the term “4-methoxyphenylacetic acid” (abbreviated as “4MPLA”) refers to a 4-O-methylated catecholamine metabolite found in normal urine. As used herein, the term “concentration” as applied to determining the concentration of the metabolite refers to a determined concentration of the metabolite. The term includes a determined concentration of the metabolite as compared to a reference (for example, a control, a reference biomarker, a baseline, or the like). In one embodiment, determining the concentration of the metabolite comprises the use of ¹H-nuclear magnetic resonance spectroscopy (abbreviated as “NMR”). In one embodiment, the method further comprises using one or more of 4MPLA, citrate, creatine ribosome, creatinine, choline, and n-acetyl-neuraminic acid as quantified in normal urine as a control.

Using the above-described methods, the expression levels of the miRNAs and the concentrations of the metabolites are determined and compared with the expression levels of the miRNAs and the concentrations of the metabolites in a normal control. As used herein, the term “normal control” refers to a subject without lung cancer. The expression levels of the miRNAs and the concentrations of the metabolites in the normal control are used as a baseline or benchmark to compare against the expression levels of the miRNAs and the concentrations of the metabolites in a subject having lung cancer or being monitored for the progression or treatment of lung cancer.

The comparison may be determined by statistical analysis, using methods known to one skilled in the art. In one embodiment, the statistical analysis comprises binary logistic regression, receiver operating characteristic curve, or both. As used herein, the term “binary logistic regression” refers to an analysis in which the observed outcome for a dependent variable can have only two possible types, “0” and “1” (which may represent, for example, “lung cancer subjects” versus “healthy controls”). As used herein, the term “receiver operating characteristic curve” (abbreviated as “ROC curve”) refers to a graph showing the true positive rate (Sensitivity) plotted as a function of the false positive rate (100-Specificity) for different cut-off points. Each point on the ROC curve represents a sensitivity/specificity pair corresponding to a particular decision threshold. The results from such analysis are indicative of whether or not the subject has lung cancer. In one embodiment, a difference in the expression levels of the miRNAs and the concentrations of the metabolites in the test subject relative to the expression levels of the miRNAs and concentrations of the metabolites of the normal control is indicative of lung cancer in the test subject.

Following a diagnosis of lung cancer, the subject may then be treated with a cancer management program to treat, ameliorate, or prevent the progression of the cancer. As used herein, the term “cancer management” refers to an effective treatment modality or program to include pharmacologic and non-pharmacologic components for treating, ameliorating, and/or preventing cancer. As used herein, the terms “treatment,” “treating,” “treat,” and the like, refer to obtaining a desired pharmacologic and/or physiologic effect. The effect can be prophylactic in terms of completely or partially preventing lung cancer or symptoms thereof and/or can be therapeutic in terms of a partial or complete cure for lung cancer and/or adverse effect attributable to lung cancer. “Treatment” covers any treatment of lung cancer in a subject, particularly in a human, and includes: (a) preventing lung cancer in a subject which may be predisposed to lung cancer but has not yet been diagnosed as having it; (b) inhibiting lung cancer, i.e., arresting its development; and (c) relieving lung cancer, i.e., causing regression of lung cancer and/or relieving one or more lung cancer symptoms. “Treatment” can also encompass delivery of an agent or administration of a therapy to provide for a pharmacologic effect. Specific treatment can be based on the knowledge of the biomarkers; for example, designer anti-miRNA oligonucleotides carried by nanoparticles can regulate the growth and suppression of cancers.

In one embodiment, assessment of the subject’s response to lung cancer treatment comprises determining the expression levels of the miRNAs and the concentrations of the metabolites prior to treatment and after treatment, comparing the expression levels of the miRNAs and the concentrations of the metabolites in a normal control, and predicting a response if there is a difference in the miRNA expression levels and metabolite concentrations.

Embodiments of the invention include not only the above method of conducting and interpreting the results of the above tests but also include related methods, and reagents, kits, assays, and the like, for conducting the tests.

In one embodiment, the invention comprises a method of analyzing for a marker indicative of lung cancer comprising:
a) obtaining a sample of serum, urine, or both from a subject suspected of having lung cancer;
b) determining in the serum sample, the expression level of one or more miRNAs;
c) determining in a urine sample, the concentration of one or more metabolites;
d) comparing the expression level of the one or more miRNAs with the expression level of the one or more miRNAs in a normal control;
e) comparing the concentration of the one or more metabolites with the concentration of the one or more metabolites in a normal control; and
f) determining whether the subject has lung cancer in accordance with the result of steps (d) and (e); wherein a difference in the expression level of the one or more miRNAs relative to the expression level of the normal control, and a difference in the concentration of the one or more metabolites relative to the concentration of the normal control, are indicative of lung cancer.

MicroRNA and metabolite “profiling” in liquid biopsy (e.g., serum, urine) may be useful as a tool for lung cancer detection, diagnosis, and prognosis, since certain miRNAs and metabolites have been found to be correlated with lung cancer. Thus, in the development of one embodiment of the present invention, it was determined whether particular serum miRNAs and urine metabolites may be indicative of NSCLC.

Based on preliminary studies described in the Examples, the inventors have identified serum microRNAs and urine metabolites which are associated with NSCLC, and may be used to detect NSCLC in a subject. The serum microRNAs and urine metabolites disclosed herein are thus diagnostic and prognostic markers of NSCLC. The method detects NSCLC using serum miRNA expression levels or urine metabolite concentrations, when taken alone or in combination.

NSCLC was detectable using only two miRNAs (miR-21, miR-223) with high sensitivity (96.4%) and specificity (88.2%). Thus, miRNA profiling may prove valuable as a complementary screening tool with low-dose computer tomography to better discriminate NSCLC lesions and reduce unnecessary interventions from false-positive results. Further, a panel of two miRNAs is more cost-effective than larger miRNA panels and facilitates screening programs on a population level. ROC analyses on each of the two miRNAs alone (miR-21, miR-223) were conducted to assess the individual ability to discriminate between NSCLC and healthy control subjects. The AUCs for the two miRNAs ranged from 0.63 and 0.79, respectively. To determine the diagnostic ability of miR-21and miR-223 in combination, a risk score analysis was conducted and the AUC improved to 0.912, with sensitivity of 96.4% and specificity of 88.2%.

Use of one metabolite (4MPLA) with NMR spectra resulted in a sensitivity of 82.1% and a specificity of 88.2%. However, the combination of the two miRNAs (miR-21, miR-223) and the single metabolite (4MPLA) was found to be far more accurate in detecting NSCLC compared to either miRNAs or metabolites, when taken alone. Early stage NSCLC patients and controls were distinguished with a sensitivity and specificity of between about 90 to about 100%.

By the method of this invention, a reliable and quick means is provided for early detection of lung cancer. Liquid biopsy for early detection of lung cancer is conveniently non-invasive, comfortable, and quick for a subject in contrast to conventional clinical practice which typically requires invasive sampling. Although sputum is a non-invasive sample, the variability in sputum quality can lower the sensitivity and specificity. Utilizing serum and urine avoid the difficulty of poor sputum sample quality. Those skilled in the art will recognize that the method of the present invention may be similarly applied to other cancers (for example, breast, gastrointestinal tract, prostate, gynecological, and head and neck cancers) and abnormal bio-physiological infections and diseases.

Based upon the identification of miRNAs and metabolites associated with lung cancer, an artificial intelligence tool was developed. In one embodiment, the artificial intelligence tool comprises an artificial neural network. As further described in Example 5, an exemplary artificial intelligence tool was developed for the early diagnosis of NSCLC using separate subsets of selected combinations of miRNA expression levels and urine metabolite concentrations from normal control and lung cancer subjects to train, validate, and test a NSCLC classification neural network algorithm. The model was thus trained using specific types, sizes, and sets of input data in multiple steps or phases. Significant amounts of time, effort, and data preparation, calculation, and optimization were thus expended to develop this machine learning solution to detect lung cancer, as further described in Examples 1-5.

In general, a standard neural network has layers which may include, for example, an input layer of processing elements, a middle layer of processing elements, and an output layer composed of a single processing element. Other embodiments of a neural network can also be used. Each of the processing elements receives multiple input signals (typically in the form of data values) which are processed to compute a single output. The output value is calculated using a mathematical equation which specifies the relationship between input data values. In the context of the present invention, a neural network is a computer simulation that produces a score based on input of one or more values for expression levels of miRNAs and one or more concentrations of metabolites for a subject. The scores produced by the network might range between 0 to 1, with scores near 0 indicating a low likelihood of lung cancer in the subject and scores near 1 indicating a high likelihood of lung cancer in the subject.

In one embodiment, the invention comprises a method for developing an artificial intelligence tool for detecting lung cancer in a subject comprising the steps of:
training a neural network with a first data set comprising known data having known values for expression levels of miRNAs and concentrations of metabolites in normal controls and lung cancer subjects;
validating the neural network by providing a second data set comprising known data having known values for expression levels of miRNAs and concentrations of metabolites in normal controls and lung cancer subjects to the neural network; and
testing the neural network by providing a third data set comprising known data having known values for expression levels of miRNAs and concentrations of metabolites in normal controls and lung cancer subjects to the neural network for analysis and determination of a score ranging between 0 and 1;
wherein the determined score near 0 indicates a low likelihood of lung cancer, or the determined score near 1 indicates a high likelihood of lung cancer.

In one embodiment, the invention comprises the artificial intelligence tool developed by the above method. As additional miRNAs and metabolites associated with lung cancer are identified over time by clinicians or researchers, the neural network may continuously “learn” through re-training or updating with the new additional data. Machine learning generally involves building a model based on mappings between possible input and desired output. The knowledge base of the neural network may thus be expanded over time to process and store historic and new additional data, thereby improving the detection of lung cancer over time as additional relevant miRNAs and metabolites are discovered. Use of the neural network may thus alleviate conventional problems associated with handling increasing volumes of data collected and used for ongoing profiling.

Training or updating the neural network with a relatively large number of miRNAs and metabolites requires the use of a computer. The invention involves computationally complex operations which would be difficult and time-consuming without computer implementation. Mental or manual calculation (i.e., pen and paper) by human effort alone would be far too arduous to provide a practical solution to the problem being solved. A lack of computer implementation would pose a major obstacle to performing the detection of lung cancer successfully and rapidly. The computer is thus required to solve this technical problem. The trained or updated artificial intelligence tool is used with a computer in a system to accelerate detection of lung cancer, thereby minimizing delay in receipt of test results and in the onset of treatment for the subject in the event that the determined score indicates a high likelihood of lung cancer.

In one embodiment, the invention comprises a system for detecting lung cancer in a subject comprising:
a computer device for inputting a data set comprising one or more values for expression levels of miRNAs and one or more concentrations of metabolites in a subject;
a neural network trained to detect lung cancer for determining a score ranging between 0 and 1 and indicative of the likelihood of lung cancer on the basis of the data set;
a display device for displaying the determined score in a human-readable form, wherein the determined score near 0 indicates a low likelihood of lung cancer or the determined score near 1 indicates a high likelihood of lung cancer.

A system generally comprises a conventional computer and processor for executing simulation of the neural network. Those skilled in the relevant art will appreciate that the invention can be practiced with other computer configurations, including mobile computing devices, such as smart phones, tablets, multiprocessor systems, microprocessor-based or programmable consumer electronics, personal computers (“PCs”), network PCs, mini- computers, mainframe computers, and the like. Computer hardware used to implement the various components, elements, modules, methods, and algorithms described herein can include a processor configured to execute one or more sequences of instructions, programming stances, or code stored on a non-transitory, computer-readable medium. The processor can be, for example, a general purpose microprocessor, a microcontroller, a digital signal processor, an application specific integrated circuit, a field programmable gate array, a programmable logic device, a controller, a state machine, a gated logic, discrete hardware components, an artificial neural network, or any like suitable entity that can perform calculations or other manipulations of data. Computer hardware can further include elements such as, for example, a memory (e.g., random access memory (RAM), flash memory, read only memory (ROM), programmable read only memory (PROM), erasable read only memory (EPROM)), registers, hard disks, removable disks, CD-ROMS, DVDs, or any other like suitable storage device or medium.

Executable sequences can be implemented with one or more sequences of code contained in a memory. In some embodiments, such code can be read into the memory from another machine-readable medium. Execution of the sequences of instructions contained in the memory can cause a processor to perform the method or steps described herein. One or more processors in a multi-processing arrangement can also be employed to execute instruction sequences in the memory. In addition, hardwired circuitry can be used in place of or in combination with software instructions to implement various embodiments described herein. Thus, the present embodiments are not limited to any specific combination of hardware and/or software.

A machine-readable medium will refer to any medium that directly or indirectly provides instructions to a processor for execution. A machine-readable medium can take on many forms including, for example, non-volatile media, volatile media, and transmission media. Non-volatile media can include, for example, optical and magnetic disks. Volatile media can include, for example, dynamic memory. Transmission media can include, for example, coaxial cables, wire, fiber optics, and wires that form a bus. Common forms of machine-readable media can include, for example, floppy disks, flexible disks, hard disks, magnetic tapes, other like magnetic media, CD-ROMs, DVDs, other like optical media, punch cards, paper tapes and like physical media with patterned holes, RAM, ROM, PROM, EPROM, and flash EPROM.

The computer system also may include input device(s) (e.g., a keyboard, mouse, touchpad, etc.) and output device(s) (e.g., a display device such as a monitor to display data and a subject’s test results, a printer for generating a hard copy of the subject’s results for the physical record, etc.). Such input device(s) and/or output device(s) provide a user interface that enables an operator (for example, a physician, nurse, or laboratory technician) to interact with the software executed by the processor.

After obtaining a biological sample from the subject and detecting the miRNAs and metabolites in the sample, the operator may input the data into the system for processing by the trained or updated neural network to output a test result. The system thus creates value for both the clinician and the subject since test results may be obtained rapidly compared to conventional screening techniques such that treatment is not delayed. In this manner, the system is integrated into a practical application, namely to effect or initiate a particular treatment for lung cancer based on test results obtained in an unconventional manner compared to standard screening techniques.

In one embodiment, the invention comprises a method for detecting lung cancer in a subject comprising:
obtaining a sample of serum, urine, or both from the subject;
determining in the serum sample, the expression level of one or more miRNAs;
determining in a urine sample, the concentration of one or more metabolites;
inputting, using a computer device, a data set comprising one or more values representing the expression level of the one or more miRNAs and the concentration of the one or more metabolites;
applying a neural network to the one or more values, wherein the neural network was trained to detect lung cancer for determining a score ranging between 0 and 1 and indicative of the likelihood of lung cancer on the basis of the data set;
displaying, using a display device, the determined score in a human-readable form, wherein the determined score near 0 indicates a low likelihood of lung cancer or the determined score near 1 indicates a high likelihood of lung cancer; and
treating the subject with a cancer management program based on the determined score.

In one embodiment, the determined score may be stored in a file in a memory or on a machine-readable medium as a subject’s electronic records. The effectiveness of the treatment may be subsequently monitored by repeating the above steps, and comparing the stored determined score with a determined score obtained posttreatment.

Embodiments of the present invention are described in the following Examples, which are set forth to aid in the understanding of the invention, and should not be construed to limit in any way the scope of the invention as defined in the claims which follow thereafter.

### Example 1 — Subject Accrual and Clinical Data Collection

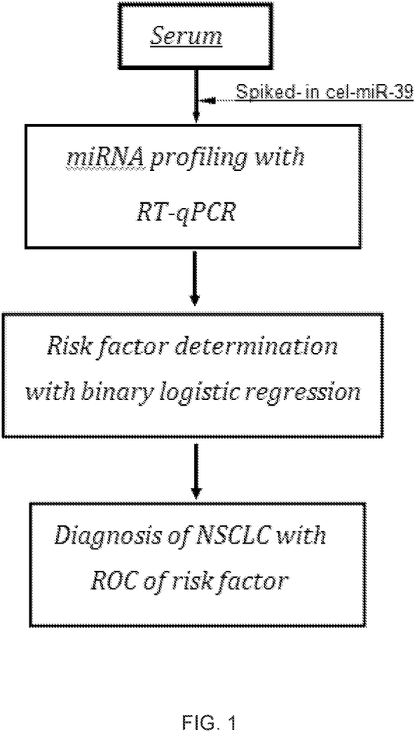

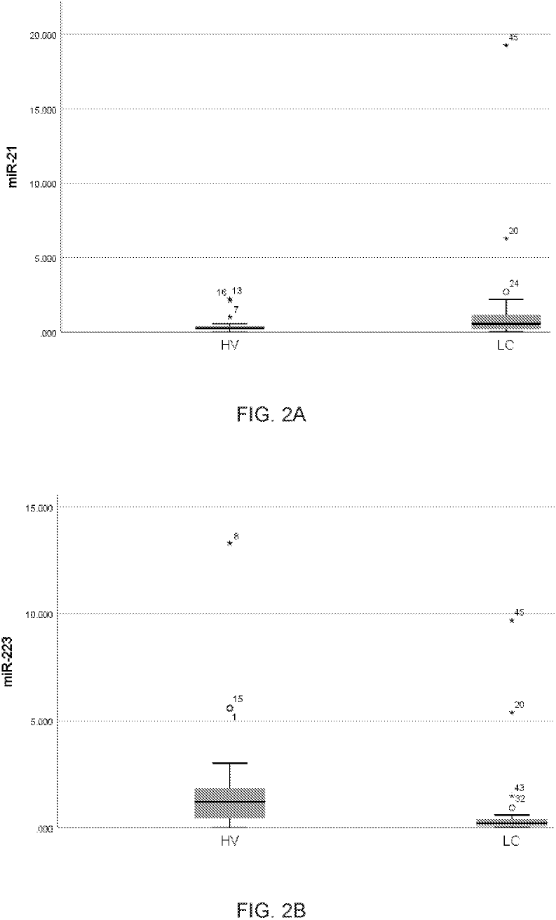

FIG. 1 shows steps taken to validate the method for detecting NSCLC in a subject using serum miRNAs and urine metabolites. Forty-five and fifty-six consecutively consenting patients and matched controls (at an approximate case to control ratio of 2:1) were accrued for serum and urine samples. Specifically, forty-six subjects with matched serum samples and fifty-six subjects with matched urine samples were accrued. A total of thirty-five subjects provided matched samples of both serum and urine. Eligibility criteria included age 18-75 years of age and life expectancy ≥ 3 months. NSCLC patients required biopsy or cytology-confirmed disease of any pathologic subtype and stage I or II disease according to the American Joint Committee on Cancer, 7^{th} edition. NSCLC patients with a prior or current history of malignancy, excluding non-melanomatous skin cancer, were excluded.

Control participants required a history of smoking, with or without chronic obstructive pulmonary disease, and included some individuals who had undergone a biopsy for what proved to be a benign lung nodule. All controls were required to have had a clinical examination and diagnostic imaging (chest X-ray or computed tomography of the chest) negative for malignancy within twelve months prior to study entry. A standard medical history was obtained. A self-reported questionnaire was used to collect demographic data, functional status and social/occupational history. Diagnostic imaging scans, pathology reports, pulmonary function test results, and previous medical and surgical history were obtained from the electronic medical record.

Table 1 displays the participant baseline characteristics and tumor characteristics. The NSCLC and control groups did not differ significantly in the group of subjects that gave serum samples and the group that had both serum and urine samples in terms of smoking history, gender and pulmonary disease. The mean age of the control group was 57 years compared to 67 years in the NSCLC group (p=0.022).

TABLE 1

Participant baseline characteristics and tumor characteristics

| Participant baseline characteristics | | NSCLC Patients | Controls | P-value |
|---|---|---|---|---|
| 45 patients with serum samples included for analysis | | | | |
| Number of participants | | n = 28 | n = 17 | |
| Age, years; median [range] | | 67 [51-76] | 57 [46-74] | 0.022 |
| Gender, n [%] | Male | 12 [42.81] | 9 [52.9] | 0.511 |
| | Female | 16 [57.2] | 8 [47.1] | |
| Current smoker, n [%] | Yes | 12 [42.9] | 8 [47.1] | 0.783 |
| | No | 16 [57.1] | 9 [52.9] | |
| Smoking history, pack-years; mean [SD] | | 42 [21.1] | 33.8 [21.3] | 0.233 |
| COPD, n [%] | Yes | 7 [25] | 1 [5.9] | 0.132 |
| | No | 21 [75] | 16 [94.1] | |
| Adenocarcinoma | | 19 [67.8] | NA | - |
| Squamous cell carcinoma | | 7 [25] | | |
| Large cell carcinoma | | 1 [3.6] | | |
| Sarcomatoid carcinoma | | 1 [3.6] | | |
| T1aN0M0 | | 7 [25] | NA | - |
| T1bN0M0 | | 5 [17.9] | | |
| T2aN0M0 | | 9 [32.1] | | |
| T2bN0M0 | | 1 [3.6] | | |
| T3N0M0 | | 2 [7.1] | | |
| T1bN1M0 | | 1 [3.6] | | |
| T2aN1M0 | | 2 [7.1] | | |
| T2bN1M0 | | 1 [3.6] | | |
| Maximum tumor diameter, cm | | | | |
| Mean [SD] | | 2.8 [1.7] | NA | - |
| Gender, n [%] | Male | 12 [57.1] | 8 [57.1] | 0.999 |
| | Female | 9 [42.9] | 6 [42.9] | |
| Current smoker, n [%] | Yes | 11 [52.4] | 6 [42.9] | 0.581 |
| | No | 10 [47.6] | 8 [57.1] | |
| Smoking history, pack-years; mean [SD] | | 42 [18.1] | 34 [22.2] | 0.170 |
| COPD, n [%] | Yes | 7 [33.3] | 1 [7.1] | 0.162 |
| | No | 14 [66.6] | 13 [92.9] | |
| Adenocarcinoma | | 16 [76.2] | NA | - |
| Squamous cell carcinoma | | 4 [19] | | |
| Large cell carcinoma | | 1 [4.8] | | |
| T1aN0M0 | | 7 [20] | NA | - |
| T1bN0M0 | | 3 [8.6] | | |
| T2aN0M0 | | 6 [17.1] | | |
| T2bN0M0 | | 1 [2.9] | | |
| T3N0M0 | | 1 [2.9] | | |
| T1bN1M0 | | 1 [2.9] | | |
| T2aN1M0 | | 1 [2.9] | | |
| T2bN1MO | | 1 [2.9] | | |
| Maximum tumor diameter, cm | | | | |
| Mean [SD] | | 2.8 [1.7] | NA | - |

Abbreviations: COPD- Chronic Obstructive Pulmonary Disease, TNM- Tumor Node Metastases, n-number, NSCLC- Non Small Cell Lung Cancer, AJCC- American Joint Committee on Cancer

### Example 2 - Serum miRNA Profiling for 45 Subjects (28 NSCLC and 17 Healthy Controls)

Serum miRNA levels were measured using quantitative real-time reverse-transcription with an exogenous control, and a comparative ΔC_{T} method was used to calculate relative miRNA expression.

For quality control, serum samples with haemolysis index (HI) > 200 were excluded. For RNA extraction, 2.6 µl of C. elegans miR-39 (cel-miR-39) (Applied Biosystems, USA, 1.6 × 10⁸ copies/µl) was spiked into each 150 µl serum sample to serve as a synthetic exogenous control. RNA from each spiked serum sample was extracted and purified using miRNeasy™ Serum/Plasma Kit (Qiagen, Canada) according to manufacturer protocols.

RNA was converted to complimentary DNA (cDNA) by reverse transcription (RT) using TaqMan™ MicroRNA Reverse Transcription Kit (Applied Biosystems, USA) and TaqMan™ MicroRNA Assay’s specific stem-loop primers (Applied Biosystems, USA). RT reactions were mixed according to manufacturer protocols under the following conditions: 16° C. for 30 mins, 42° C. for 30 mins, 85° C. for 5 mins, hold at 4° C. Expression levels of miRNAs (miR-21, miR-223 and the control, cel-miR-39) were quantified using Real-Time PCR analysis under the following cycling conditions: 95° C. for 10 min, followed by 40 cycles of 95° C. for 15 second and 60° C. for 1 min (7900HT Fast Real-Time PCR System, Applied Biosystems, USA). Cycle threshold (C_{T}), defined as the number of PCR cycles required for a fluorescent signal to be higher than baseline variability, was determined using SDS 2.3 software (Applied Biosystems, USA). Using cel-miR-39 as the control, relative expression levels of serum miRNAs were calculated and represented by 2-^{ΔCT}, where ΔC_{T}= C_{T} (miRNA of interest) – C_{T} (cel-miR-39).

The expression levels of miR-21 and miR-223 were analyzed using binary logistic regression and receiver operating characteristic (ROC) curve. The combined effect of miR-21 and miR-223 allowed for a model of risk threshold determination with a weighted combination of miR-21 and miR-223.

A genomic variable from miRNA binary logistic regression analysis (genomVAR) was thus generated to express a subject’s disease status as follows:

$$\text{genomVAR} = 0.840 + 2.389 {\text{RQ}}_{\text{miR21}} \text{- 2} {\text{.70RQ}}_{\text{miR223}} \qquad \text{(1)}$$

where RQ represents RNA quantification data from quantitative real-time reverse-transcription polymerase chain reaction, and the coefficients indicate the weighted contributions of miR-21 and miR-223. The selection of miR-21 and miR-223 for genomVAR was based on the opposite bio-functional contributions in formula (1), which correlate with the regulatory mechanisms of miR-21 and miR-223 for cancer. Specifically, miR-21 is reported to be associated with up-regulation of oncogenes, while miR-223 is associated with tumor suppressor genes.

A ratio variable genomic ratio of miRNAs (genomRAT) of miR-21 to miR-223 for detection of NSCLC was expressed as follows:

$$\text{genomRAT =} {\text{RQ}}_{\text{miR21}} / {\text{RQ}}_{\text{miR223}} \qquad \text{(2)}$$

where RQ represents RNA quantification data from quantitative real-time reverse-transcription polymerase chain reaction for miR-21 and miR-223.

The genomVAR and genomRAT were used to identify NSCLC and healthy controls by using ROC curves. genomRAT was directly calculated from the C_{T} values of miR-21 and miR-223 (C_{TmiR21}-C_{Tm}i_{R223}).

The exogenous control cel-miR-39 did not affect genomRAT as a ratio. Cel-miR-39 was detectable by RT-qPCR for all forty-five subjects, and 200 copies was the lower limit of detection (C_{T}= 35.29 ± 0.36). There was no significant difference (p=0.59) in C_{T} between the NSCLC group (C_{T}=28.38 ± 0.725) and the control group (C_{T}=28.51 ± 0.78), and thus spiked-in cel-miR-39 was validated as a suitable exogenous control. This is a significant improvement for negating the potential effects of various endogenous/exogenous controls or housekeeping genes, and for significantly decreasing the cost and workload for RT-qPCR assays.

Both miRNAs of interest (miR-21 and miR-223) were detectable by RT-qPCR in all forty-five subjects. Table 2 shows the miR-21 and miR-223 expressions in NSCLC subjects and cancer-free controls.

TABLE 2

Expression level and receiver operating characteristic analysis of two micro-RNAs in non-small cell lung cancer patients and controls

| miRNA | NSCLC patients (n=28) | | Controls (n=17) | | AUC in ROC |
|---|---|---|---|---|---|
| | Mean | Std Dev | Mean | Std Dev | |
| miR-21 | 1.591 | 3.679 | 0.524 | 0.665 | 0.63 (95% confidence interval: 0.47-0.80) |
| miR-223 | 0.795 | 2.020 | 2.312 | 3.300 | 0.79 (95% confidence interval: 0.64-0.95) |

Abbreviations: NSCLC- Non Small Cell Lung Cancer, AUC- Area Under the Curve, ROC- Receiver Operating Characteristics, n-number

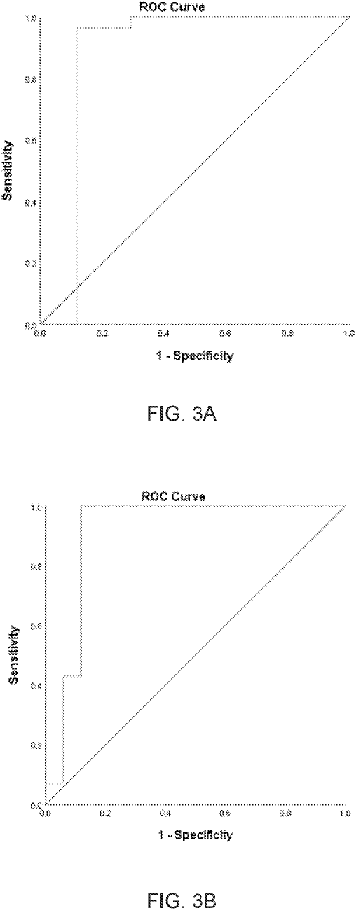

The ROC analysis of genomVAR was used to differentiate NSCLC and cancer-free controls (FIG. 3B). Applying binary logistic regression and ROC yielded an AUC of 0.912 (0.792-1.0). A cut-off point of 0.4944 differentiated NSCLC and controls with 96.4% sensitivity (27/28) and 88.2% specificity (15/17).

The ROC analysis of genomRAT was used to differentiate NSCLC and cancer-free controls (FIG. 3A). The simplified variable reached the same level of performance as genomVAR, with an AUC of 0.876 (95%: 0.723-1.0). A cut-point of 0.8060 differentiated NSCLC and controls with 96.4% sensitivity (27/28) and 88.2% specificity (15/17).

Given the relatively small sample size, internal validation of miRNA data was conducted using k-fold cross-validation. The data were divided into three parts (k=3). The cut points were determined on the training set by repeating the ROC five times on a randomly chosen training set. The results were averaged to yield the final values. The cut point was determined on the training set and validated using the test set. The AUC for the training set was 86.46% with a sensitivity of 94.4% and specificity of 92.0%. The AUCs for the training set and the test set with the cut point (0.91297) were 89.26% and 92.98%, respectively.

### Example 3 - Urine Metabolite Profiling for 56 Subjects (32 NSCLC and 24 Healthy Controls)

The relative concentrations of six urine metabolites were analyzed to assess the best correlations with cancer. 4-Methoxyphenylacetic acid (4MPLA) and citrate were used as covariates for binary logistic regression owing to their statistical significance (Table 3). The metabolite model process was as follows:

$$\begin{matrix}\text{Metabolomics variable} \left(\text{metabVAR}\right) = \text{-0} \text{.828 + 1} {\text{.588C}}_{\text{4MPLA}} \text{-} \\ 1.103 {\text{C}}_{\text{Citrate}}\end{matrix} \qquad \text{(3)}$$

The relative urinary concentrations of 4MPLA in patients and healthy controls were measured using ¹H-nuclear magnetic resonance (NMR) spectroscopy. Urine samples were centrifuged at 12000 rpm for 15 min to remove cells and other precipitated materials. For NMR analysis, 400 µL of clear urine was mixed with 200 µL of buffer solution (KH₂PO₄ 1.5 M in D₂O) containing 0.1 % TSP-d4 as a chemical shift reference and 2 mM sodium azide (NaN₃) as bacteriostatic agent. The pH was adjusted to 7.00 by adding drops of 4 M solutions of KOD or DCI. Samples were then centrifuged at 12000 rpm for 10 min and 550 µL of each solution was transferred to a 5 mm NMR tube.

NMR spectra were acquired on a Bruker Avance DRX-600 spectrometer operating at 600.27 MHz and 300 K for H observation (Carrola et al., 2011; Beckonert et al., 2007). For each sample, a standard 1D ¹H NMR spectrum was acquired, using a water suppression pulse sequence with water irradiation during relaxation delay and mixing time (‘noesypr1d’ in Bruker library, SW 10330.58 Hz, TD 32 K data points, relaxation delay 4 s, mixing time 100 ms, 2000 scans). All spectra were processed with a line broadening of 0.3 and a zero filling factor of 2, with automatic phasing and baseline corrected. The chemical shifts were referenced internally to the TSP signal at δ 0.00. The chemical shift (peak position) and intensity were automatically determined by TopSpin™ (V3.2, Bruker, Germany). Intensity of peaks was normalized to the sum of the total spectral integral (δ 8.5-0.50 excluding δ 4.55-6.05) to enable comparison between samples. The intensity of the strongest peak of the targeted metabolite was used as the apparent and relative concentration. This concentration of each sample was then used as the variable for the case and control.

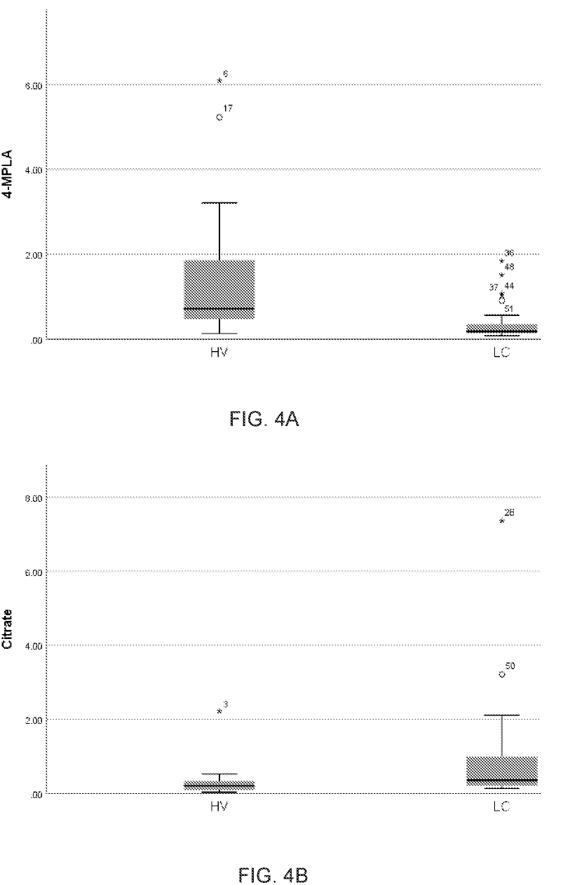

The urine metabolites of 4-methoxyphenylacetic acid (4MPLA), citrate, creatine ribosome, creatinine, choline, and n-acetyl-neuraminic acid (NANA) were selected as potential reference biomarkers to discriminate lung cancer subjects and healthy controls. Table 3 sets out the descriptive statistics of the metabolites and the AUC of ROC. Only 4MPLA demonstrated a significant difference (p<0.05), whereas the other metabolites did not show significant differences. The mean concentration of 4MPLA for lung cancer subjects was significantly lower than the healthy controls (p=0.001) (FIG. 4A). The mean concentration of citrate for lung cancer subjects was higher than the healthy controls (p=0.062).

TABLE 3

Statistical results of six targeted metabolites for lung cancer and healthy control

| Metabolite | Lung cancer (32) mean ± std | Healthy control (24) mean ± std | P-value | AUC |
|---|---|---|---|---|
| 4MPLA | 0.380±0.429 | 1.349±1.539 | 0.001 | 0.83 (95% Cl: 0.71-0.94) |
| Citrate | 0.863±1.383 | 0.303±0.431 | 0.061 | 0.73 (95% Cl: 0.59-0.86) |
| Creatine ribosome | 0.271±0.566 | 0.662±1.208 | 0.112 | 0.68 (95% Cl: 0.53-0.82) |
| Creatinine | 0.137±0.157 | 0.170±0.180 | 0.458 | 0.70 (95% Cl: 0.57-0.84) |
| Choline | 0.208±0.131 | 0.250±0.169 | 0.298 | 0.57 (95% Cl: 0.42-0.73) |
| NANA | 0.193±0.597 | 0.066±0.024 | 0.304 | 0.59 (95% Cl: 0.43-0.74) |

Abbreviations: 4MPLA-4-methoxyphenylacetic acid, NANA- n-acetyl-neuraminic acid, AUC- Area under the Curve

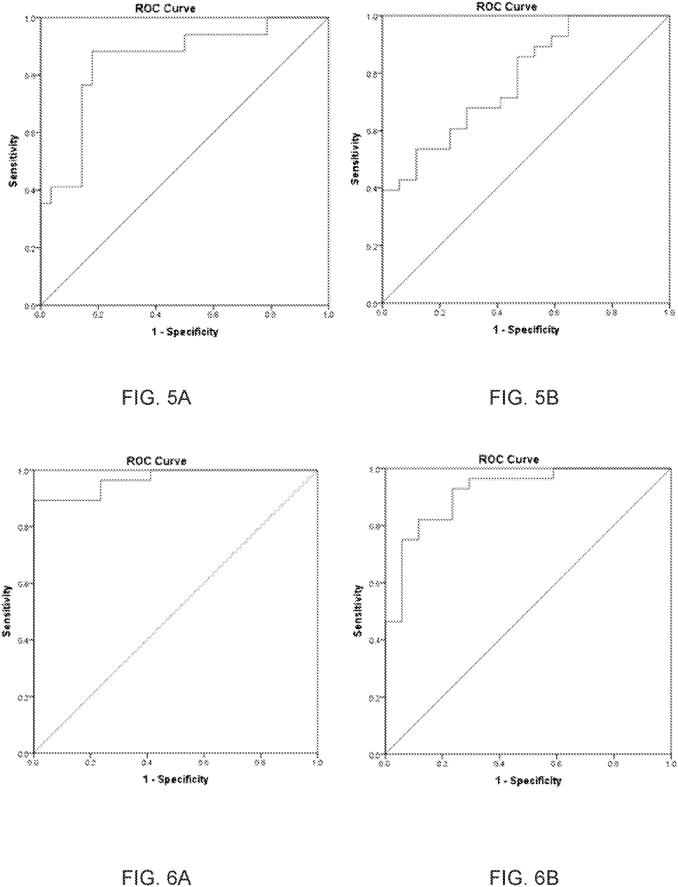

The ROC of 4MPLA demonstrated a sensitivity of 82.1% and a specificity of 88.2% (FIG. 5A). The ROC of citrate demonstrated a lower sensitivity of 67.9% and a specificity of 70.6% (FIG. 5B). FIGS. 5A-B show the ROC results with 4MPLA and citrate. Upon application of the binary logistic methodology with formula (3) and variable metabVAR, the ROC of metabVAR showed a sensitivity of 87.5% and a specificity of 71.9%. The AUC of ROC was 0.827 (95%: 0.713-1.00).

With regard to the metabolomics ratio of 4MPLA and citrate, a cut-point of -0.5661 for the metabVAR demonstrated a sensitivity of 87.5% and a specificity of 71.9% to differentiate NSCLC from control samples, and the AUC of the ROC was noted to be 0.827 (95%: 0.713-1.00).

A metabolomics ratio (metabRAT) of 4MPLA and citrate was created as a simple ratio to discriminate NSCLC:

$$\text{metabRAT} = {\text{C}}_{\text{4MPLA}} / {\text{C}}_{\text{Citrate}} \qquad \text{(4)}$$

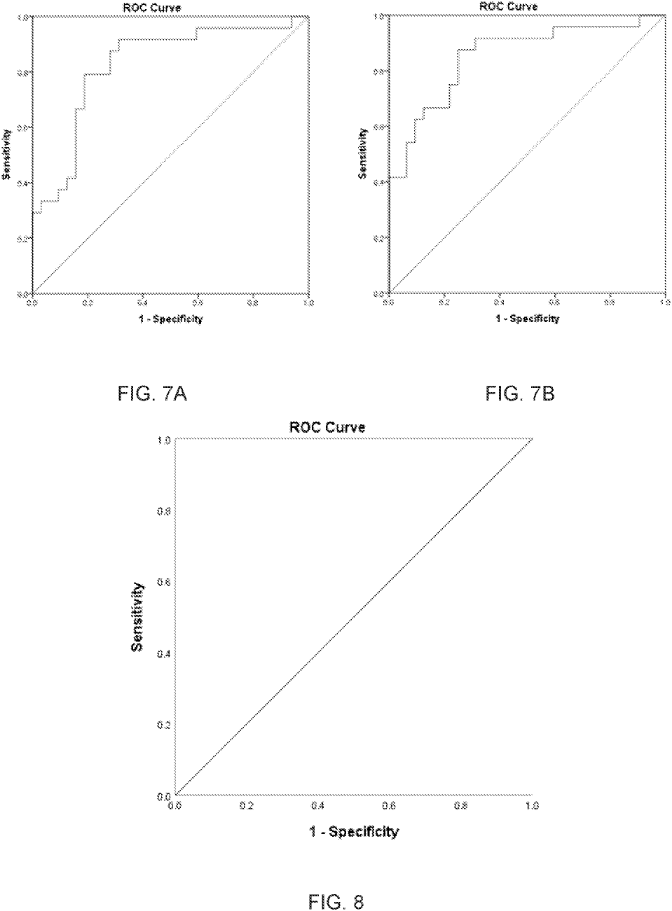

A cut-point of 1.3641 for the metabRAT demonstrates a sensitivity of 87.5% and a specificity of 75% to differentiate NSCLC from controls, and the AUC of ROC is 0.854 (95%: 0.751-0.958) (FIGS. 7A-B).

In a manner similar to the internal validation of the miRNA data, internal validation of the metabolomics data was conducted using k-fold cross-validation. Since the sample size was relatively small, the data were divided into three parts (k=3). The cut points were determined on the training set by repeating the ROC five times on a randomly chosen training set. The results were averaged to yield the final values. The cut point determined on the training set for 4MPLA was 0.405476 and validated using the test set. The AUC for the training set was 0.826 (95% CI: 0.713-0.938), with a sensitivity of 87.5% and specificity of 78.1%. The AUCs for the training set and the test set with the above cut points were 79.2% and 87.5%, respectively.

### Example 4 - Combined miRNA Profiling and Metabolomics for 35 Subjects (21 NSCL and 14 Healthy Controls)

Micro-RNA expression levels and metabolite concentrations were combined to assess accuracy in detecting lung cancer. Although miRNA profiling and metabolomics originated from different sample sources (serum versus urine) and were measured using different analytical platforms (RT-qPCR versus NMR), miRNA expression levels and metabolite concentrations are continuous variables which correspond to the biological state of the subject’s body, including its oncological related mechanisms.

A mathematical model was established from the combined miRNA profiling of miR-21 and miR-223 and metabolomics data using the single metabolite 4MPLA to further improve detection. Binary logistic regression was used to test miRNA profiling plus metabolomics, resulting in a combined variable from miRNA (comboVAR) expressed as follows:

$$\begin{matrix}\text{comboVAR = 87} \text{.32 + 346} {\text{.29RQ}}_{\text{miR21}} \text{- 412} {\text{.60RQ}}_{\text{miR223}} - \\ 53.72 {\text{C}}_{\text{4MPLA}}\end{matrix} \qquad \text{(4)}$$

A combined ratio of miRNA expression level and metabolite concentration (comboRAT) was expressed as follows:

$$\text{comboRAT} = \text{-} \left(2.362 + 0.036 \text{genomRAT - 0} \text{.840metabRAT}\right)$$

(5)

The comboRAT was used in the ROC method to determine a cut-off point based on the outcome of interest (lung cancer subjects versus healthy controls). The cut-off point was selected based on Yoden’s index (maximum sensitivity and maximum specificity) obtained from the ROC curve. The cut-off point was used to categorize the comboRAT variable into high risk and low risk.

Based on the 35 patients who provided both serum & urine samples, the ROC of comboVAR identified a cut-point of 1.361 that could accurately differentiate NSCLC from controls with a sensitivity of 100% and a specificity of 100%, and an AUC of 1.00. Although this combined model provided accurate diagnosis for lung cancers, it required data from both genomic (RT-qPCR) and metabolomics (NMR). FIG. 8 shows the ROC curve for comboVAR. Addition of a further metabolite beyond 4MPLA to the analysis was not deemed to be of any additional benefit.

For a similar application using the comboRAT with genomRAT and metabRAT, the ROC of comboVAR yielded a sensitivity of 82.1% and a specificity of 81.9%. The AUC of ROC was 0.918 (95%: 0.836-1.00). FIGS. 6A-B show the ROC curves for comboVAR and comboRAT.

Internal validation of combining miRNA expression levels and urine metabolite concentrations was conducted using k-fold cross-validation. Since the sample size was small, the data were divided into three parts (k=3). The cut point was determined on the training set by repeating the ROC five times on a randomly chosen training set. The results were averaged to yield the final values. The cut point which was determined on the training set was -1.24675 and validated using the test set. The AUC for the training set was 92.62 with a sensitivity of 100% and specificity of 86.7%. The AUCs for the training set and the test set with the cut point were 92.9% and 85.7%, respectively.

In summary, ROC analysis of the miRNA expression alone yielded a sensitivity of 96.4% and a specificity of 88.2% for the detection of early stage NSCLC, with AUC = 0.91 (CI 95%: 0.80-1.0). Relative urinary concentrations of 4MPLA and citrate were significantly different between NSCLC case and healthy control (p = 0.008). The ROC analysis of 4MPLA yielded a sensitivity of 82.1% and a specificity of 88.2%, with AUC = 0.85. The composite process combining miRNA and metabolite expression demonstrated a sensitivity and specificity of nearly 100.0% and AUC = 1.

### Example 5 - Artificial Intelligence Tool for Diagnosis of Early Stage NSCLC

Development of an artificial intelligence tool for the early diagnosis of NSCLC involved using separate subsets of selected combinations of miRNA expression levels and urine metabolite concentrations to train, validate, and test a NSCLC classification neural network algorithm.

The algorithm was generated using the TensorFlow™ open source software library, which is an open source machine learning library that can be used to undertake various numerical computations. The machine learning is an Artificial Neural Network that uses both input and output data to develop predictive models. The Network is composed of layers that filter (convolve or enclose in groups) the inputs to obtain useful information. The convolutional layers have parameters that are learned and are automatically adjusted to extract the most useful information. A set of data was initially used to train the software to recognize outcomes, which were then validated with a new set of data. The validated neural network was then given a third study set to analyze and predict the possible outcome.

The Artificial Neural Network was given a training set of data (Table 4) from selected combinations of miRNA expression levels and urine metabolite concentrations data from 16 individuals, both controls and cancer patients, with an AUC set at 1 to predict the outcome of interest (lung cancer subjects versus healthy controls). A 3 × 3 Neural Network with a Rectified Linear Activation Unit (Relu) Activation Function was used. Hyperparameter optimization to configure the convolution layers was explored using the Bayesian method. The machine learning was then verified using an additional validation study set of data from 7 other individuals (Table 5). Lastly, a separate test data set involving data from 11 individuals (Table 6) was submitted for evaluation, and the Artificial Neural Network yielded a sensitivity of 100% and a specificity of 100%, and an AUC of 1.00 for predicting the presence of cancer, all using the same selected combinations of miRNA expression levels and urine metabolite concentrations.

TABLE 4

Training Data

| Patient ID | miR21 | miR223 | 4Methoxyphenylaceticacid | citrate | State | Prediction |
|---|---|---|---|---|---|---|
| 33 | 0.9960 | 0.5960 | 0.5670 | 0.2747 | 1 | 0.9984 |
| 3 | 1.2842 | 0.9420 | 1.0334 | 0.4474 | 1 | 0.9962 |
| 22 | 0.3270 | 0.4970 | 0.1780 | 0.3909 | 0 | 0.3333 |
| 11 | 0.2720 | 1.1322 | 0.5970 | 0.2244 | 0 | 0.0215 |
| 5 | 1.0224 | 1.8463 | 0.5750 | 0.3035 | 0 | 0.0142 |
| 32 | 0.3940 | 5.5733 | 2.0579 | 0.1966 | 0 | 0.0001 |
| 10 | 0.2630 | 5.6091 | 0.7460 | 0.2573 | 0 | 0.0001 |
| 7 | 2.1189 | 0.3960 | 0.1390 | 0.1953 | 1 | 1.0000 |
| 14 | 0.9850 | 0.2330 | 0.1450 | 1.7390 | 1 | 1.0000 |
| 4 | 0.1940 | 0.4200 | 0.4750 | 0.1395 | 0 | 0.0444 |
| 24 | 0.2200 | 0.3530 | 0.1100 | 0.3644 | 1 | 0.6946 |
| 20 | 0.5570 | 13.3100 | 0.1350 | 0.0929 | 0 | 0.0000 |
| 28 | 0.2260 | 1.6594 | 3.2152 | 0.0577 | 0 | 0.0016 |
| 12 | 0.7480 | 0.1080 | 0.1890 | 0.2277 | 1 | 0.9997 |
| 25 | 0.2160 | 0.1200 | 1.8426 | 1.4210 | 1 | 0.9998 |
| 17 | 0.0997 | 0.0125 | 0.1120 | 0.3446 | 1 | 0.9855 |

TABLE 5

Validation Data

| Patient ID | miR21 | miR223 | 4Methoxyphenylaceticacid | citrate | State | Prediction |
|---|---|---|---|---|---|---|
| 31 | 0.3340 | 0.4640 | 0.4390 | 0.3601 | 0 | 0.3003 |
| 26 | 0.4310 | 0.0754 | 0.0832 | 0.7927 | 1 | 0.9999 |
| 13 | 0.0071 | 1.7191 | 0.6970 | 0.0368 | 0 | 0.0073 |
| 19 | 0.2560 | 0.0842 | 1.0766 | 0.3645 | 1 | 0.9337 |
| 30 | 0.1480 | 0.6850 | 1.7369 | 0.4373 | 0 | 0.0232 |
| 23 | 0.0579 | 0.3570 | 0.6430 | 0.1548 | 0 | 0.0415 |
| 2 | 0.4920 | 0.3580 | 0.1590 | 0.2967 | 1 | 0.9775 |

TABLE 6

Test Data

| Patient ID | miR21 | miR223 | 4Methoxyphenylaceticacid | citrate | State | Prediction |
|---|---|---|---|---|---|---|
| 21 | 19.2482 | 9.6993 | 0.9130 | 0.1513 | 1 | 1.0000 |
| 18 | 0.9100 | 0.2020 | 0.1290 | 3.2163 | 1 | 1.0000 |
| 27 | 0.2210 | 1.2150 | 5.2310 | 0.0409 | 0 | 0.0005 |
| 15 | 1.3099 | 0.2110 | 0.1190 | 7.3641 | 1 | 1.0000 |
| 8 | 0.2450 | 0.2260 | 0.2920 | 0.2170 | 1 | 0.7493 |
| 0 | 0.1910 | 1.7450 | 1.9898 | 0.1798 | 0 | 0.0041 |
| 6 | 6.2829 | 5.3985 | 0.3720 | 0.1415 | 1 | 0.9994 |
| 16 | 1.0340 | 0.1120 | 0.1460 | 1.2050 | 1 | 1.0000 |
| 1 | 0.0541 | 0.0281 | 0.1880 | 0.4281 | 1 | 0.9860 |
| 9 | 0.7800 | 0.2300 | 0.3330 | 1.3005 | 1 | 1.0000 |
| 29 | 0.2000 | 0.0551 | 0.2220 | 0.2227 | 1 | 0.9513 |

### Example 6 - Statistical Analysis

Descriptive statistics were obtained for the study variables. Mean and standard deviations were reported for normally distributed continuous variables and median (range) were reported for non-normally distributed continuous variables. Frequency and proportions were reported for categorical variables. Two categorical variables were compared using Chi-square tests. Fisher’s exact tests were reported when the cell frequencies were less than 5. Mann-Whitney test was used to compare the continuous variables between two groups. Binary logistic regression and receiver operator characteristic curve were used to discriminate lung cancer subjects and healthy controls. The cut-off point obtained from the ROC curve was selected based on sensitivity and specificity. Statistical analysis was conducted using SPSS version 22 software (IBM SPSS, USA). A p-value <0.05 was used for all statistical comparison and two-tailed tests were used.

### Example 7 - Results

Discussed below are results obtained in connection with the experiments of Examples 1-6.

The present invention relates to a non-invasive process using only body fluid samples for the early detection of lung cancer which could dramatically alter the current conventional clinical practice. The results demonstrate that using a targeted panel of two miRNAs from serum samples and one targeted urine metabolite can distinguish between patients with early stage NSCLC and controls with a sensitivity and specificity between 90-100%. The use of two miRNAs alone gave a sensitivity of 96.4% and a specificity of 88.2% to detect NSCLC which is similar to or better than previously reported larger miRNA panels (Boeri et al., 2011; Sozzi et al., 2014; Wang et al., 2015; Nadal et al., 2015; Gyoba et al., 2017, 2018). A panel of two miRNAs is more cost-effective than larger miRNA panels and would facilitate screening programs on a population level.

The ROC analyses of the individual miRNAs were conducted to assess the individual ability to discriminate between NSCLC and control subjects. The AUCs for miRNA-21 and miRNA-223 ranged from 0.63 and 0.79, respectively (Table 2). To determine the diagnostic ability of miR21 and miR223 in combination, a risk score analysis was conducted and the AUC improved to 0.91, with sensitivity of 96.4% and specificity of 88.2%.

Synthetic spiked-in cel-miR-39 was utilized as miRNA exogenous control, and thus ensured effective miRNA extraction, cDNA synthesis, and PCR amplification. Cel-miR-39 avoids the issue of frequent hemolysis that can occur with miR-16 as an endogenous control (Navarro et al., 2011; Kirschner et al., 2011), and the problem of extensive RNAse-mediated degradation with using U6 snRNA (Xiang et al., 2014).

Although metabolomics has attracted attention owing to its close relevance to phenotype (Emwas et al., 2013), it is also challenging due to the sheer number of different molecules present in biofluids like urine (Moldovan et al., 2014), and at present only about 220 metabolites have been identified and quantitated by NMR or LC-MS (Bouatra et al., 2013). In cancer diagnosis by urine NMR metabolomics, only a limited number of metabolites can be quantified by untargeted metabolomics (Carrola et al., 2011), due to the significant overlap of NMR spectra of the different biomolecules, which makes untargeted metabolomics a complex challenge. Hence targeted metabolomics is the way forward, and this study identified that the relative urinary concentrations of 4MPLA and citrate were significantly different between NSCLC case and matched control, making them potential targets for use in detection of NSCLC.

A composite methodology showed unique results: the miRNA expression alone yielded a sensitivity of 96.4% and a specificity of 88.2% for the detection of early stage NSCLC, with AUC = 0.91 (Cl 95%: 0.80-1.0), whereas ROC analysis of the metabolite 4MPLA alone yielded a sensitivity of 82.1% and a specificity of 88.2%. The composite methodology of miRNA/4MPLA with internal validation yielded a sensitivity of 100.0% and a specificity of 86.7%. Evaluation of the data using machine-learning further improved our findings of detecting early stage NSCLC with sensitivity and specificity of close to 100%.

Without being bound by any theory, the invention may be a landmark step towards early non-invasive detection of lung cancers. The low cost of running the tests and the ease of scaling down the metabolomics testing to point-of-care devices are other potential areas that can have a significant impact on routine uptake of this panel. The invention may likely improve the early detection of early lung cancers and have a clinical impact by increasing the likelihood of providing curative treatment and prolonged survival.

It should be apparent, however, to those skilled in the art that many more modifications besides those already described are possible without departing from the inventive concepts herein. The inventive subject matter, therefore, is not to be restricted except in the scope of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

### REFERENCES

All publications mentioned are incorporated herein by reference (where permitted) to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Beckonert, O., Keun, H.C., Timothy MD Ebbels, et al. Metabolic profiling, metabolomic and metabonomic procedures for NMR spectroscopy of urine, plasma, serum and tissue extracts, Nature Protocol, 2007, 2(11): 2692-2703.

Berindan-Neagoe I, Monroig Pdel C, Pasculli B, Calin GA. MicroRNAome genome: a treasure for cancer diagnosis and therapy. CA Cancer J Clin. 2014 Sep-Oct; 64(5):311-36.

Bianchi F, Nicassio F, Marzi M, Belloni E, Dall’olio V, Bernard L, Pelosi G, Maisonneuve P, Veronesi G, Di Fiore PP: A serum circulating miRNA diagnostic test to identify asymptomatic high-risk individuals with early stage lung cancer. EMBO Mol Med; 3(8): 495-503, 2011.

Boeri M, Verri C, Conte D, Roz L, Modena P, Facchinetti F, Calabrò E, Croce C, Pastorino U, Sozzi G: MicroRNA signatures in tissues and plasma predict development and prognosis of computed tomography detected lung cancer. Proc Natl Acad Sci USA 108(9): 3713-3718, 2011.

Bouatra S, Aziat F, Mandal R, et al, The human urine metabolome. PLoS One. 2013 Sep 4;8(9):e73076. doi: 10.1371/journal.pone.0073076.

Calin GA, Croce CM: MicroRNA signatures in human cancers. Nat Rev Cancer 6(11): 857-866, 2006.

Carrola J, Rocha CM, Barros AS., Metabolic signature of lung cancer in biofluids: NMR-based metabonomics of urine, J. Proteome Res, 2011 Jan 7, 10(1): 221-30.

Eder M, Scherr M: MicroRNA and lung cancer. N Engl J Med 352(23): 2446-2448, 2005.

Emwas, AH.M., Salek, R.M., Griffin, J.L. et al. NMR-based metabolomics in human disease diagnosis: applications, limitations, and recommendations Metabolomics (2013) 9: 1048.

Gyoba J, Roa W, Guo L, Ghosh S, Bedard EL. Lung Cancer Risk Score Analysis Using Plasma microRNA Profiles. Journal of Thoracic Oncology. 2017;12(11).

Gyoba J. Translational Application of microRNA Profiling to Detect Non-Small Cell Lung Cancers [Thesis]: University of Alberta; 2018.

Kim JO, Gazala S, Razzak R, Guo L, Ghosh S, Roa WH, Bédard EL: Non-small cell lung cancer detection using microRNA expression profiling of bronchoalveolar lavage fluid and sputum. Anticancer Res 35(4): 1873-1880, 2015.

Kirschner MB, Kao SC, Edelman JJ, et al. Haemolysis during sample preparation alters microRNA content of plasma. PLoS One 6(9): e24145, 2011.

Klupczynska A, Dereziński P, Garrett TJ, et al Study of early stage non-small-cell lung cancer using Orbitrap-based global serum metabolomics. J Cancer Res Clin Oncol. 2017 Apr;143(4):649-659.

Mazzone PJ, Wang XF, Beukemann M, et al Metabolite Profiles of the Serum of Patients with Non-Small Cell Carcinoma. J Thorac Oncol. 2016 Jan;11(1):72-8.

Moldovan L, Batte KE, Trgovcich J, et al: Methodological challenges in utilizing miRNAs as circulating biomarkers. J Cell Mol Med18(3):371-390, 2014.

Nadal E, Truini A, Nakata A, Reddy RM, Chang AC, Ramnath N, Gotoh N, Beer DG, Chen G: A Novel Serum 4-microRNA Signature for Lung Cancer Detection. Sci Rep 5: 12464, 2015.

Navarro A, Diaz T, Gallardo E, et al : Prognostic implications of miR-16 expression levels in resected non-small-cell lung cancer. J Surg Oncol 103(5): 411-415, 2011.

Razzak R, Bedard E, Kim J, Gazala S, Guo L, Ghosh S, Joy A, Nijjar T, Wong E, Roa WH: The prospective evaluation of stage-dependent sputum micro-RNA expression profiles for the detection of non-small cell lung cancer. Curr Oncol 23(2): e86-e94, 2016.

Roa WH, Kim JO, Razzak R, Du H, Guo L, Singh R, Gazala S, Ghosh S, Wong E, Joy AA, Xing JZ, Bedard EL: Sputum microRNA profiling: a novel approach for the early detection of non-small cell lung cancer. Clin. Invest Med 35(5): E271, 2012.

Rocha CM, Carrola J, Barros AS, et al, Metabolic signature of lung cancer in biofluids: NMR-based metabonomics of blood plasma, J Proteome Res, 2011 Sep 2; 10 (9): 4314-24.

Shen J, Liao J, Guarnera MA, Fang H, Cai L, Stass SA, Jiang F: Analysis of MicroRNAs in sputum to improve computed tomography for lung cancer diagnosis. J Thorac Oncol 9(1): 33-40, 2014.

Sozzi G, Boeri M, Rossi M, Verri C, Suatoni P, Bravi F, Roz L, Conte D, Grassi M, Sverzellati N, Marchiano A, Negri E, La Vecchia C, Pastorino U: Clinical Utility of a Plasma-Based miRNA Signature Classifier Within Computed Tomography Lung Cancer Screening: A Correlative MILD Trial Study. J Clin Oncol 32(8): 768-773, 2014.

Wang C, Ding M, Xia M, Chen S, Van Le A, Soto-Gil R, Shen Y, Wang N³, Wang J, Gu W, Wang X, Zhang Y, Zen K, Chen X, Zhang C, Zhang CY: A Five-miRNA Panel Identified From a Multicentric Case-control Study Serves as a Novel Diagnostic Tool for Ethnically Diverse Non-small-cell Lung Cancer Patients; EBioMedicine 2(10): 1377-1385, 2015.

Weiss GJ, Bemis LT, Nakajima E, Sugita M, Birks DK, Robinson WA, Varella-Garcia M, Bunn PAJr, Haney J, Helfrich BA, Kato H, Hirsch FR, Franklin WA: EGFR regulation by microRNA in lung cancer: correlation with clinical response and survival to gefitinib and EGFR expression in cell lines. Ann Oncol 19(6): 1053-1059, 2008.

Wozniak MB, Scelo G,Muller DC, Mukeria A, Zaridze D, Brennan P: Circulating MicroRNAs as Non-Invasive Biomarkers for Early Detection of Non-Small-Cell Lung Cancer; PLoS One 10(5): e0125026, 2015.

Xiang M, Zeng Y, Yang R, et al: U6 is not a suitable endogenous control for the quantification of circulating microRNAs. Biochem Biophys Res Commun 454(1):210-214, 2014.

Xing L, Su J, Guarnera MA, Zhang H, Cai L, Zhou R, Stass SA, Jiang F: Sputum microRNA biomarkers for identifying lung cancer in indeterminate solitary pulmonary nodules. Clin Cancer Res 21(2): 484-489, 2015.

What is claimed is:

1. A method of detecting lung cancer in a subject comprising the steps of:
a) determining in a serum sample, the expression level of one or more miRNAs;
b) determining in a urine sample, the concentration of one or more metabolites;
c) comparing the expression level of the one or more miRNAs with the expression level of the one or more miRNAs in a normal control;
d) comparing the concentration of the one or more metabolites with the concentration of the one or more metabolites in a normal control;
e) determining whether the subject has lung cancer in accordance with the result of steps (c) and (d); wherein a difference in the expression level of the one or more miRNAs relative to the expression level of the normal control, and a difference in the concentration of the one or more metabolites relative to the concentration of the normal control, are indicative of lung cancer; and
f) treating the subject with a cancer management program based on the determination in step (e).

2. The method of claim 1, wherein only steps (a), (c), (e), and (f) are conducted.

3. The method of claim 1, wherein only steps (b), (d), (e), and (f) are conducted.

4. The method of claim 1, wherein the lung cancer comprises non-small cell lung carcinoma.

5. The method of claim 4, wherein the one or more miRNAs comprise miR-21 and miR-223.

6. The method of claim 5, wherein the step of determining the expression level of the one or more miRNAs comprises a real-time reverse transcription-quantitative polymerase chain reaction (RT-qPCR) assay.

7. The method of claim 6, further comprising using Caenorhabditis elegans miR-39-5p as a control.

8. The method of claim 5, wherein the one or more metabolites comprise 4-methoxyphenylacetic acid.

9. The method of claim 8, wherein the step of determining the concentration of the one or more metabolites comprises ¹H-nuclear magnetic resonance spectroscopy.

10. The method of claim 9, further comprising using one or more of 4-methoxyphenylacetic acid, citrate, creatine ribosome, creatinine, choline, and n-acetylneuraminic acid as quantified in normal urine as a control.

11. The method of claim 1, wherein the steps (c) and (d) comprise statistical analysis selected from binary logistic regression, receiver operating characteristic curve, or both.

12. The method of claim 11, wherein the steps (c) and (d) comprise a mathematical algorithm to express oncogenic and cancer-suppressive characteristics of the miRNAs, metabolites, or both based on their biological functional pathways.

13. The method of claim 1, further comprising assessing one or more parameters selected from demographic data, clinical characteristics, functional status, social/occupational history, diagnostic imaging scans, pathology reports, pulmonary function test results, previous medical and surgical history, age, gender, history of smoking, and presence of chronic obstructive pulmonary disease.

14. The method of claim 1, further comprising assessing the subject’s response to lung cancer treatment comprising determining the expression levels of the miRNAs and the concentrations of the metabolites prior to treatment and after treatment, comparing the expression levels of the miRNAs and the concentrations of the metabolites in a normal control, and predicting a response if there is a difference in the levels.

15. A method of analyzing for a marker indicative of lung cancer comprising:
a) obtaining a sample of serum, urine, or both from a subject suspected of having lung cancer;
b) determining in the serum sample, the expression level of one or more miRNAs;
c) determining in a urine sample, the concentration of one or more metabolites;
d) comparing the expression level of the one or more miRNAs with the expression level of the one or more miRNAs in a normal control;
e) comparing the concentration of the one or more metabolites with the concentration of the one or more metabolites in a normal control; and
f) determining whether the subject has lung cancer in accordance with the result of steps (d) and (e); wherein a difference in the expression level of the one or more miRNAs relative to the expression level of the normal control, and a difference in the concentration of the one or more metabolites relative to the concentration of the normal control, are indicative of lung cancer.

16. A method for developing a tool for detecting lung cancer in a subject comprising the steps of:
training a neural network with a first data set comprising known data having known values for expression levels of miRNAs and concentrations of metabolites in normal controls and lung cancer subjects;
validating the neural network by providing a second data set comprising known data having known values for expression levels of miRNAs and concentrations of metabolites in normal controls and lung cancer subjects to the neural network; and
testing the neural network by providing a third data set comprising known data having known values for expression levels of miRNAs and concentrations of metabolites in normal controls and lung cancer subjects to the neural network for analysis and determination of a score ranging between 0 and 1;
wherein the determined score near 0 indicates a low likelihood of lung cancer, or the determined score near 1 indicates a high likelihood of lung cancer.

17. A neural network tool developed by the method of claim 16.

18. A system for detecting lung cancer in a subject comprising:
a computer device for inputting a data set comprising one or more values for expression levels of miRNAs and one or more concentrations of metabolites in a subject;
a neural network trained to detect lung cancer for determining a score ranging between 0 and 1 and indicative of the likelihood of lung cancer on the basis of the data set;
a display device for displaying the determined score in a human-readable form, wherein the determined score near 0 indicates a low likelihood of lung cancer or the determined score near 1 indicates a high likelihood of lung cancer.

19. A method for detecting lung cancer in a subject comprising:
obtaining a sample of serum, urine, or both from the subject;
determining in the serum sample, the expression level of one or more miRNAs;
determining in a urine sample, the concentration of one or more metabolites;
inputting, using a computer device, a data set comprising one or more values representing the expression level of the one or more miRNAs and the concentration of the one or more metabolites;
applying a neural network to the one or more values, wherein the neural network was trained to detect lung cancer for determining a score ranging between 0 and 1 and indicative of the likelihood of lung cancer on the basis of the data set;
displaying, using a display device, the determined score in a human-readable form, wherein the determined score near 0 indicates a low likelihood of lung cancer or the determined score near 1 indicates a high likelihood of lung cancer; and
treating the subject with a cancer management program based on the determined score.
